(19) United States
(12) Patent Application Publication
(10) Pub. No.: US 2007/0093452 A1
(43) Pub. Date: Apr. 26, 2007

(54) Use of bivalent or polyvalent trisaccharides as fusion-inhibitors in all HIV types, subtypes, groups, strains, and circulating recombinant forms

(76) Inventor: Mercedes Farquharson, Monroe, NC (US)

Correspondence Address: Mercedes T. Farquharson, 3018 Hampton Downs Drive, Monroe, NC, 28112, US

(21) Appl. No.: 11/253,985

(22) Filed: Oct. 20, 2005

(51) Int. Cl. A61K 31/715

(52) U.S. Cl. 514/61

(57) ABSTRACT

This invention relates to compositions containing bivalent or polyvalent trisaccharides, specifically but not exclusively, melezitose and raffinose, and to their use in the treatment/prevention/cure of HIV and AIDS. In particular, such compositions can be used to competitively inhibit formation of the viral fusion complex. This invention also relates to compositions containing bivalent or polyvalent trisaccharides, specifically but not exclusively, melezitose and raffinose, and to their use in other viral infections such as Influenza and Ebola, and furthermore to their use in the treatment of cancer. This invention also relates to the identification of a formerly unidentified “density” present in the Phe43 cavity of HIV. This invention hereby identifies said density as cholesterol.

### CROSS REFERENCE TO RELATED APPLICATIONS

Not Applicable

### STATEMENT REGARDING FEDERALLY SPONSORED RESEARCH OR DEVELOPMENT

Not Applicable

### REFERENCE TO A SEQUENCE LISTING, A TABLE, OR A COMPUTER PROGRAM LISTING COMPACT DISC APPENDIX

Not Applicable

### BACKGROUND OF THE INVENTION

Human Immunodeficiency Virus (HIV) is a retrovirus that invades cells in the human immune system in order to replicate itself. It primarily infects CD4+ T lymphocytes. The HIV replication cycle begins when the glycoprotein envelope gp120 on the surface of the virus links to a CD4 glycoprotein receptor on the host cell. A trans-membrane subunit of the viral membrane, gp41, mediates fusion of the viral membrane and the cell membrane.

Once attached, the virus commences to replicate itself, whilst shortening the lifespan of the CD4 host cell. Ultimately, viral replication exceeds the immune system's ability to replenish CD4 cells, at which point immunologic function declines and Acquired Immunodeficiency Syndrome (AIDS) develops.

When gp120 links with the CD4 receptor, conformational changes in gp120's structure take place, one of which is the formation of a cavity on gp41 known as the Phe43 insertion cavity. The cavity is a pocket where CD4 binds and interacts with two of the complex and one of the high-mannose sugar branches on the rim of the binding cavity.

The Phe43 cavity was discovered in 1997, when Structural Biologist, Dr. Kim of Whitehead Laboratories, who is a member of the AIDS Vaccine Research Committee of the National Institutes of Health, discovered the core structure of gp41 using X-ray crystallography. (“Core Structure of gp41 from the HIV Envelope Glycoprotein,” 1997, April 18 issue of Cell (89:263-273)) Kim described the cavity as extending deep into the hydrophobic interior of gp120, as being roughly spherical, and as having a diameter of 8A°. (“Structure of an HIV gp120 envelope glycoprotein in complex with the CD4 receptor and a neutralizing human antibody” Kwong, D et al; Nature, Vol 393, 18 Jun., 1998)

The cavity is called the Phe43 cavity because the phenyl ring of CD4's Phenylalanine 43 forms a lid over it after the virus has attached to the target cell. An important attribute of the Phe43 cavity is that it is well conserved even during HIV mutation, which has made it an obvious target for drug discovery and antibody design.

One of the most challenging obstacles to finding a cure for HIV has been its rapid and continual mutation. In 1998, Dr. Josheph Sodroski stated that the challenge [to HIV treatment] is to generate neutralizing antibodies that see things that are conserved. (“HIV-1 Envelope Structure” Committee Meeting Minutes May, 1998 New York, N.Y. NIAID Division of AIDS)

Kim stated, “Our structure, combined with data from other laboratories, suggests that a small molecule constructed specifically to block [interaction between the Phe43 cavity and Phe43 of CD4] could stop fusion and prevent the virus from entering cells.”

Sodroski commented that “it is difficult to imagine a drug that would work after the cavity is revealed, because there is only a tiny little space left for a small molecule to penetrate, and almost no time left for the small molecule to penetrate the cavity and interrupt what takes place next, i.e. HIV penetrating the cell.” (“HIV-1 Envelope Structure” ibid)

It follows then that a suitable molecule would need to induce the same conformational change in gp120 that is induced by CD4's Phe43, thereby causing gp120 to expose the Phe43 cavity. Once the cavity was exposed, the molecule would enter the cavity, “plug” it, and make it inaccessible to the oligosaccharide heads of the glycosphingolipid surface on the target cell. Such a molecule would be effective against all known strains of HIV.

Melezitose is a molecule that matches these requirements. Melezitose, also known as O-α-D-Glucopyranosyl-(1->3)-β-D-fructofuranosyl-α-D-glucopyranoside, has the formula C18 H32 O16. It is a bivalent trisaccharide comprised of 2 glucose molecules and 1 fructose molecule. Its molecular weight is 504.44. (Merck 12,5860; ebi_id 6018)

Melezitose induces the same conformational change in gp120 that exposes the Phe43 cavity, and it fits into the Phe43 cavity like a hand in a glove. Once the cavity is exposed, melezitose enters the cavity, “plugs” it, and makes it inaccessible to Phe43 of CD4.

Another suitable molecule is raffinose (β-D-Fructofuranosyl-O-α-D-galactopyranosyl-(1->6)-α-D-glucopyranoside), (also called gossypose, melitose, or melitriose), with the formula C18 H32 O16. Raffinose is a bivalent trisaccharide comprised of 1 molecule each of D-galactose, D-glucose, and D-fructose. The molecular weight of raffinose is 504.44; (Merck 12,8279; ebi id 6028)

Of these two trisaccharides, melezitose confers the highest anti-HIV potential. All analogues of these two trisaccharides may be used in HIV/AIDS treatment and prevention. Examples include but are not limited to:
1. α1-3, α1-6-D-mannotriose, C18 H32 O16, FW 504.44
2. Glcα1-3Glcα1-3Glc nigerotriose, C18 H32 O16, FW 504.44
3. Linear B-6 Trisaccharide Galα1-3Galβ1-4Glc C₁₈H₂₂O₁₆ FW 504.44 C₁₈ H₃₂ O₁₆ FW 504.4
4. α1-3, β1-4 Galactotriose Galα1-3Galβ1-4Gal C₁₈H₃₂O₁₆ FW 504.44
5. Laminaritriose

The centre of the Phe43 cavity is dominated by a large spherical “density”, which is over 4A° from any protein atom. “The size, shape and predicted hydrogen bonding of this density are inconsistent with those expected for water, isopropanol, ethylene glycol, or any of the other major crystallization components.” (Kwong, D et al) As yet, said density has not been identified. This invention identifies said density as cholesterol.

### BRIEF SUMMARY OF THE INVENTION

The present invention claims that bivalent or polyvalent trisaccharides, or other compounds having the formula C18 H32 O16 and/or a molecular weight of approximately 504 Daltons are bioavailable inhibitors of HIV entry into permissive cells, and can be used as therapeutic agents in the treatment and prevention of HIV and AIDS.

Moreover, the present invention relates to a method of preventing infections caused by HIV-1 by administering bivalent or polyvalent trisaccharides alone or in combination. The present invention relates also to a method of treating established retroviral infections by administering trisaccharides alone or in combination.

Also, the present invention relates to a composition comprising at least one bivalent or polyvalent trisaccharide, or a combination of at least two bivalent or polyvalent trisaccharides. The bivalent or polyvalent trisaccharide may be, for example, melezitose or raffinose.

The present invention also identifies the previously unidentified density present in the Phe43 cavity as being cholesterol.

### DETAILED DESCRIPTION OF THE INVENTION

a) Definitions

As used herein, the term “bivalent” means having a valance of two. “Polyvalent” means having a valence of 3 or higher. A “trisaccharide” is any of a variety of carbohydrates that yield three monosaccharide molecules on complete hydrolysis. Examples of the bivalent or polyvalent trisaccharides of the present invention which may be used in the treatment and prevention of HIV infection include the trisaccharide melezitose and the trisaccharide raffinose. It should be noted, however, that the use of any bivalent or polyvalent trisaccharide that has the ability to inhibit binding of viral gp120 to the cellular target(s) is considered to fall within the scope of the present invention. Melezitose is solvent.

b) How the bivalent or polyvalent trisaccharides prevent binding.

The antiviral activity of the bivalent or polyvalent trisaccharides of the present invention occurs because said trisaccharides serve as alternative receptors for the viral gp120. Specifically, the unconjugated trisaccharides act as decoys and bind with the viral gp120 thereby preventing HIV fusion with the target cell.

b) Why bivalent or polyvalent trisaccharides work (using melezitose as an example):

1) Melezitose is the perfect size to enter all the way into the mouth of the Phe43 cavity.
2) It is widely known that hydrophobic cavities with steroidal bases, such as the Phe43 cavity, can bind saccharides.
3) There are water molecules inside the Phe43 cavity. When melezitose enters the cavity, it will absorb the water molecules present in the cavity. After absorbing the water molecules, melezitose will swell and fill the entire Phe43 cavity.
4) The water retentive qualities of melezitose are unsurpassed among sugars.
   b) Other Uses for Melezitose

Though this invention pertains to HIV, Melezitose and other bivalent or polyvalent trisaccharides have an application to other viruses, including, but not limited to, influenza and Ebola. Said trisaccharides also have an application to Cancer.

c) Conclusion

In conclusion, the subject matter of the present invention relates to compositions comprising at least one bivalent or polyvalent trisaccharide, specifically melezitose or raffinose, and a pharmaceutically acceptable carrier, which can be used to competitively inhibit formation of the viral fusion complex, and thereby prevent and/or treat infections caused by all HIV types, subtypes, groups, strains, and circulating recombinant forms.

Moreover, the present invention also relates to said compositions and said pharmaceutically acceptable carriers, which can be used to competitively inhibit formation of any viral fusion complex, caused by, for example, but not limited to, Influenza and Ebola. It also relates to said compositions and said pharmaceutically acceptable carries which can be used in the treatment of Cancer.

Finally. the subject matter of the present invention also relates to the identity of the previously unidentified density present in the Phe43 cavity, and hereby identifies said density as cholesterol.

1. A method of inhibiting the fusion of a retrovirus with cell membranes, comprising the step of administering a composition comprising one or more bivalent or polyvalent trisaccharides to a mammal in an amount sufficient to effect said inhibition of fusion.

2. The method of claim 1 wherein the retrovirus is a Human Immunodeficiency Virus (HIV).

3. The method of claim 1, wherein said composition comprises one or more of said bivalent or polyvalent trisaccharides in an amount sufficient to synergistically augment said inhibition of fusion.

4. A method of preventing an infection in a mammal caused by a retrovirus, comprising the step of administering a composition comprising one or more bivalent or polyvalent trisaccharides to a mammal, wherein said composition is administered in an amount sufficient to effect said prevention.

5. A method of treating an infection in a mammal caused by a retrovirus, comprising the step of administering a composition comprising one or more bivalent or polyvalent trisaccharides to a mammal, wherein said composition is administered in an amount sufficient to effect said treatment.

6. The method of claim 4 or 5 wherein said retrovirus is a Human Immunodeficiency Virus (HIV).

7. A method for preventing transmission of HIV in a mammal, comprising the step of administering a composition comprising one or more bivalent or polyvalent trisaccharides to said mammal, wherein said composition is administered in an amount sufficient to prevent said transmission.

8. The method of claim 7, wherein the transmission is perinatal vertical transmission.

9. A composition comprising one or more bivalent or polyvalent trisaccharides, wherein one or more of said bivalent or polyvalent trisaccharides inhibits interaction of CD4 receptors, viral gp120 and membrane glycolipids.

10. The composition of claim 9 wherein at least one said bivalent or polyvalent trisaccharide is selected from the group consisting of melezitose and raffinose.

11. The composition of claim 9 wherein said composition is selected from the group consisting of a pharmaceutical composition and a nutritional composition.

12. The composition of claim 9 wherein said composition can be administered by a route selected from the group consisting of parenteral administration, enteral administration, and dermal administration.

13. The composition of claim 9 wherein said parenteral administration is intravenous.

14. The composition of claim 9 wherein said enteral administration is oral.

15. The composition of claim 9 wherein said dermal administration is local.

16. claims 1 through 15 as they may apply to Influenza, Ebola, or other viral infections besides HIV.

17. claims 1 through 15 as they may apply to Cancer instead of HIV.

18. I also claim that the previously unidentified large spherical “density” in the centre of the Phe43 cavity, which is over 4A° from any protein atom, is cholesterol.
